(19) United States
(12) Patent Application Publication
(10) Pub. No.: US 2010/0291649 A1
(43) Pub. Date: Nov. 18, 2010

(54) CONTROL OF CONTAMINANT YEAST IN FERMENTATION PROCESSES

(75) Inventors: ETHAN BARUCH SOLOMON, Wilmington, DE (US); DERRICK OKULL, Wilmington, DE (US)

Correspondence Address: E I DU PONT DE NEMOURS AND COMPANY;LEGAL PATENT RECORDS CENTER, BARLEY MILL PLAZA 25/1122B, 4417 LANCASTER PIKE, WILMINGTON, DE, 19805, US

(73) Assignee: E. I. DU PONT DE NEMOURS AND COMPANY, Wilmington, DE (US)

(21) Appl. No.: 12/467,728

(22) Filed: May 18, 2009

(51) Int. Cl. C12P 7/06; C12N 1/16

(52) U.S. Cl. 435/161; 435/255.1

(57) ABSTRACT

A fermentation process for the production of ethanol from natural sources, such as corn, comprising introducing a fermentable sugar, an inoculant yeast, and a stabilized chlorine dioxide into a fermentation system is disclosed. The stabilized chlorine dioxide is added preventatively to the fermentation system to substantially prevent growth of contaminating microorganisms such as contaminant yeast.

### FIELD OF THE INVENTION

The present invention relates to a fermentation process to produce ethanol, specifically, a process to control contaminant yeast in the fermentation process.

### BACKGROUND OF THE INVENTION

As petroleum reserves become depleted and more expensive, the need for alternative, and preferably sustainable, energy sources increases. For some years, ethanol has been considered and has been used as an option for partial or complete replacement of petroleum-based fuels for different applications. Ethanol-powered automobiles are a reality. Ethanol has advantages over the use of conventional gasoline as a renewable fuel source.

Ethanol is a major chemical product which has been produced by humans for millennia from natural sources. Currently both industrial ethanol (e.g., fuel) and beverage ethanol are produced on large scale from natural sources by fermentation processes in which sugar is converted to ethanol and carbon dioxide by inoculant yeast. Many feedstocks can be used to provide the sugar for fermenting. Current natural sources include corn, milo, wheat, barley, millet, straw, sorghum, sugar cane, sugar beets, molasses, whey, and potatoes. In fact, any starch or cellulosic material, which includes nearly all plants, can be used as a source of sugar for use in producing ethanol, as starch or cellulose can be a precursor to sugar.

Generally, biocides perform poorly in fermentation systems, because they are non-specific and can also attack inoculant yeast in addition to target bacteria. Stabilized chlorine dioxide (SCD) is a biocide that has been used in fermentation systems to treat bacterial infection. SCD can be used to prevent bacterial infection as disclosed in WO 2007/149450. Repeated additions may also be required as indicated above.

Contamination by undesirable yeast such as wild yeast is a severe problem in the ethanol industry. In particular, wild yeast are a persistent contaminant in beverage ethanol production. Furthermore, wild yeast contamination is an issue for the yeast and yeast extract industries.

The great majority of ethanol is produced by strains of the inoculant yeast Saccharomyces cerevisiae. Strains of S. cerevisiae with characteristics that are important for ethanol production (rapid growth, tolerance to elevated temperature, tolerance to high osmotic stress, high ethanol production) are sold into the industry by a number of suppliers.

Basilio, et al. (Current Microbiology (2008), 56, 322-326) found that yeasts from the genera Dekkera, Picchia, Hansenula, Candida, and Zygosaccharomyces among many others, and even wild Saccharomyces are often found in both industrial and beverage ethanol production. These unwanted microorganisms are introduced into the process through the feedstock, process water, air, operators, and numerous other sources. The presence of these yeasts can cause severe episodes of contamination. Unchecked, wild (contaminant) yeast can account for more than 30% of the total yeast biomass in a fermentation process, resulting in reduced ethanol productivity.

In plants employing continuous operations, contamination by undesirable yeast, hereinafter referred to as contaminant yeast, which includes “wild yeast” is a grave threat. Continuous plants rely on the inoculant yeast in their primary fermenter to continuously grow and produce ethanol. These plants rarely propagate new batches of inoculant yeast and often operate for months without the addition of fresh inoculant yeast into fermentation. Since raw feedstocks often contain high levels of other species of yeast (i.e., contaminant yeast), plants that operate in continuous mode are highly susceptible to contamination.

In a continuous fermentation, the process is designed to sustain the inoculant yeast in a perpetual logarithmic growth phase to allow a steady state in which feedstock is continually introduced and ethanol is continually produced. When undesired yeast contaminate the process, the contaminant yeast can overwhelm the system by gradually dominating the inoculant yeast until a new inoculum must be introduced to re-establish the population of the inoculant yeast. Alternatively, an un-scheduled shut down may be necessary. In processes, including batch processes, in which yeast is recycled to begin new fermentation, the impact of contaminant yeast can be compounded as the population of contaminant yeast tends to increase with each successive cycle.

At present, there is no widely used method for the control of contaminant yeast contamination in industrial ethanol production.

Ziegler discloses in WO 2007/097874 a method to reduce undesirable microorganism contaminants in a fermentation process by generating chlorine dioxide (ClO₂) gas, dissolving in an aqueous solution and introducing the solution into a stream containing an undesirable microorganism, a fermentable carbohydrate or cellulose, and a desirable microorganism, such as yeast. The undesirable microorganism can be spoilage bacteria, contaminant yeast or killer yeast. This process requires generation equipment and reactants necessary to generate ClO₂ gas. The generated ClO₂ must be used as it is produced, because it degrades when exposed to light, or when in contact with any organic matter such as would be present in a fermentation process.

While bacterial infections can be controlled with antibiotics, there is no antibiotic (fungicide) known to selectively inhibit contaminant yeasts such as Dekkera without severely affecting the performance of the inoculant S. cerevisiae.

Sulfitation (the addition of sulfite) is used in the wine industry to non-selectively control yeast following the completion of fermentation. Sulfite is considered a necessary but undesired contaminant in finished product. Sulfitation is unlikely to be practical in fuel-ethanol because it is non-selective and because of the low sulfur requirements in the finished products.

Polyhexamethyl biguanide (PHMB) has been used experimentally to differentially inhibit contaminant yeast, while having little effect on fermenting S. cerevisiae. High levels of anionic molecules in fermentation would almost certainly neutralize the biocidal properties of PHMB, which is dependent on its cationic charge to be effective, thus, reducing its effectiveness. In addition, PHMB is expensive, and thus is also unlikely to be of significant industrial value because of the cost.

There remains a need to control microorganisms including bacteria and contaminant yeast in the ethanol industry, while having little or no effect on the fermenting yeasts or ethanol production. In addition there is a need for a solution to control contaminant yeast that reduce the efficiency of fermentation. The method must be simple to apply and does not require generation equipment or several components to be effective, but can be applied in a single step. The present invention meets these needs.

### SUMMARY OF THE INVENTION

The present invention provides a process to control and substantially prevent the growth of undesirable yeast. In one embodiment, there is provided a process to substantially impede the growth of contaminant microorganism in a reaction system for production of products derived from the metabolism of a nutrient source by an inoculant. For example, yeast may be used in the production of enzymes and in the production of ethanol by a fermentation process. A process of this invention to substantially impede the growth of contaminant microorganism in a reaction system for production of products derived from the metabolism of a nutrient source by an inoculant microorganism comprises introducing an inoculant microorganism and a stabilized chlorine dioxide solution into the reaction system.

In a particular embodiment, there is provided a process to substantially impede the growth of contaminant yeast in a fermentation system, without significantly impeding the growth of inoculant yeast. That is, the contaminant yeast is prevented from growing and dominating the fermentation system at the expense of the inoculant yeast. This embodiment comprises introducing a fermentable sugar, an inoculant yeast, and a stabilized chlorine dioxide solution into a fermentation system wherein the inoculant yeast converts the sugar into ethanol and carbon dioxide. The stabilized chlorine dioxide is added to one or more of the fermentable sugar, the inoculant, or other operations of the fermentation system in an amount effective to impede the growth of contaminant yeast. Other operations include operations such as heat exchangers, yeast inoculant propagation tanks, primary fermentation tanks, secondary fermentation tanks, saccharification tanks etc. By impeding the growth of contaminant yeast, this process prevents the contaminant yeast from negatively impacting the inoculant yeast. Based on the dose rate, a reduction in the population of the inoculant yeast may occur, as can be seen in Example 2. However, at the end of a typical incubation, the population of the inoculant yeast is not negatively impacted by treatment with SCD relative to no treatment, whereas in contrast, the population of the contaminant yeast is greatly reduced.

In this process, the amount of stabilized chlorine dioxide added is generally from about 0.0001% (1 part per million (ppm) to about 5% (50,000 ppm), based on the weight of activated chlorine dioxide which can be produced and the total weight of the fermentation system.

Alternatively, the stabilized chlorine dioxide can be, for example, added in production of yeast and yeast products via propagation. Thus, there is further provided a process to substantially impede the growth of contaminant yeast in a yeast or yeast product comprising introducing stabilized chlorine dioxide into a yeast propagation process.

### DETAILED DESCRIPTION OF THE INVENTION

The present invention relates to the production of several products as a result of fermentation or similar processes, including, in particular, the production of ethanol, both industrial ethanol and beverage ethanol via fermentation; the production of yeast and yeast products via propagation; the production of enzymes and other by-products resulting from the metabolism of a nutrient source by microorganisms such as yeast and fungi. There is a process comprising introducing a nutrient source, an inoculant yeast, and a stabilized chlorine dioxide solution into reaction system wherein the inoculant yeast metabolizes the nutrient source. This process may be performed to increase yeast population, that is, to undergo propagation, or to produce a by-product of the metabolic process, such as an enzyme.

In one particular embodiment, there is provided a process to substantially impede or prevent the growth of undesirable yeast, in a fermentation system or in a propagation system. In one particular embodiment, the process comprises introducing a fermentable sugar, an inoculant yeast, and a stabilized chlorine dioxide solution into a fermentation system wherein the inoculant converts the sugar into ethanol and carbon dioxide; and wherein the stabilized chlorine dioxide is added to one or more of the fermentable sugar, the inoculant yeast, or other operations of the fermentation system in an amount effective to decrease the growth of the undesirable microorganisms.

### Components

### Nutrient Source/Fermentable Sugar

By nutrient source, it is meant a source of elements essential to the survival of the inoculant yeast or other inoculant microorganism, for example sources of carbon, nitrogen, oxygen, sulfur, phosphorus, magnesium, and a variety of trace elements such as metals. The carbon source can be a fermentable sugar, an alcohol such as methanol, or a hydrocarbon such as octane. The nitrogen source can be urea, an ammonium salt or a nitrate. A salt such as magnesium sulfate can provide magnesium and sulfur. Phosphorus can be supplied as a sodium or potassium salt, while oxygen can be obtained by directly introducing air into the process. Other elements can be added directly or are naturally present in process components.

A fermentable sugar is a common nutrient source. A fermentable sugar suitable for use in this invention can be derived from essentially any plant source comprising sugar, starch and/or cellulose. That is, starch and/or cellulose can be converted by processes known in the art, e.g., using enzymes, to sugar suitable for use as a fermentable sugar in this invention. The fermentable sugar can be derived from one or more of any grain-based product such as corn, wood chips, wheat straw, corn stover, switch grass. The fermentable sugar may alternatively be derived from milo, barley, millet, sorghum, sugar cane, sugar beets, molasses, whey, potatoes. Processes are known to those skilled in the art to convert these sources to fermentable sugar.

Conveniently, the fermentable sugar is derived from corn, using either the wet mill or dry mill process. In a wet mill process, corn is soaked or steeped and then separated into components. In a dry mill process, corn is ground into meal and processed without separation. The corn starch component from the wet mill process or meal from the dry mill process is mixed with water and enzymes and cooked to solubilize the starch.

Corn starch is a polysaccharide, that is, a polymer made of individual units of glucose. The corn starch is converted to smaller (shorter) polysaccharides, i.e., dextrins, by enzymes (a-amylase). The smaller polysaccharides are converted to glucose (monosaccharide) using the enzyme glucoamylase, thus forming the fermentable sugar.

As an alternative to corn, the fermentable sugar can be derived from molasses. Molasses can be obtained from a variety of sources including sugar cane or sugar beets, for example, as a byproduct of the process to manufacture crystalline sugar. Molasses is typically obtained as a syrup, to which other ingredients may be added in preparation for fermentation. These other ingredients include sugarcane juice, beet juice, water, and vitamins or other nutrients. Whether one or more of the other ingredients are added and the amount added will vary in a molasses-derived fermentable sugar.

The term “mash” is used to herein to refer to a composition comprising a fermentable sugar. Mash includes any mixture of mixed grain or other fermentable carbohydrates in water used in the production of ethanol at any stage from mixing of the fermentable sugar in water to prior to any cooking and saccharification through to completion of fermentation, as defined in Jacques, K. A., Lyons, T. P., Kelsall, D. R, “The Alcohol Textbook”, 2003, 426-424, Nottingham University Press, UK.

In a fermentation process, sugar is typically present in the fermentation system in a concentration of about 5 to about 40% (weight/volume), preferably in the range of about 10 to 35% (weight/volume).

### Inoculant Yeast

For purposes herein, an inoculant yeast is a yeast which has been deliberately selected for a particular conversion in a reaction system. For example, an inoculant yeast may be selected for production of additional yeast in a yeast propagation (such as for use as baker's yeast); an inoculant yeast may be selected for metabolism of a particular nutrient source for production of enzymes. In specific application, an inoculant yeast is selected for fermentation of a fermentable sugar to produce ethanol of desired quality and in desired quantity. Inoculant yeasts are generally selected because of their ability to completely ferment available sugars, tolerance to high levels of osmotic stress, elevated temperatures, and high concentrations of ethanol. Yeast are commonly used in ethanol fermentation. Yeast are microorganisms capable of living and growing in either aerobic (with oxygen) or anaerobic (lacking oxygen) environments. For purposes herein, the yeast that is used in the fermentation process is referred to herein as an (the) “inoculant” yeast to distinguish from “contaminant” yeast, such as wild yeast, which are contaminant yeast in the process. The inoculant yeast is also referred to herein as a “desirable” yeast. Suitable inoculant yeasts for use in the process of this invention include, but are not limited to Saccharomyces cerevisiae (S. cerevisiae), Saccharomyces uvarum (S. uvarum), Schizosaccharomyces pombe (S. pombe), and Kluyveromyces sp.

The inoculant yeast may be introduced into the fermentation system as a yeast inoculum or activated inoculant yeast. That is, prior to introducing inoculant yeast into a fermenting vessel or other feed stream to a fermentation system, a yeast inoculum is produced. A yeast inoculum is produced by charging a yeast starter culture and a nutrient composition to a propagation tank. The nutrient composition may comprise one or more fermentable sugar, enzyme, and water to grow or activate the yeast. The propagation tank is a separate vessel from the fermentation system. While it is recognized that yeast propagation can occur in the fermentation vessel during the fermenting process, activation of yeast in a propagation tank provides a highly active inoculant yeast. Thus, highly active yeast is introduced to the fermenting vessel.

For continuous fermentation processes, there is often no separate yeast propagation tank. Yeast may or may not be recycled in a continuous process. When no recycle is available, yeasts grow and produce ethanol continuously in a stage called a primary fermentation. When recycle is available, such as is common when molasses or sugarcane juice is used as a feedstock for the fermentable sugar, yeasts are recycled by separation from the other fermentation components, (usually using centrifugation) and typically treated with acid in a separate tank (generally called a yeast recycle tank) to condition the yeast cells for a new fermentation cycle. Alternatively, the yeast may be separated from other fermentation components, then dried and sold as a co-product. These steps are well known to those skilled in the art.

Inoculant yeast is generally added to the fermentation system in an amount typically about 1×10⁵ to 1×10⁷ cells per gram of fermentation components. It will be recognized by those skilled in the art that this amount may vary depending on the method of fermentation employed.

### Contaminant Yeast

Fermentation processes do not always eliminate unwanted microorganisms prior to the introduction of desired organisms, in this case, selected species of inoculant yeast, such as S. cerevisiae. The undesired species of yeast are commonly referred to as contaminant yeast. They include species such as Brettanomyces, Saccharomyces, Dekkera, Picchia, Hansenula, Candida, and Zygosaccharomyces among many others. These yeast are naturally present in feedstocks commonly used in fermentation. Contaminant yeasts consume valuable nutrients in the fermentation medium, and do not produce ethanol of desired quality or at desired concentrations. Furthermore, certain species of contaminant yeast produce compounds that negatively affect the inoculant yeast, thus reducing the efficiency of the process or product quality.

### Stabilized Chlorine Dioxide

The term “stabilized chlorine dioxide” as used herein means one or more chlorine dioxide-containing oxy-chlorine complexes and/or one or more chlorite-containing components and/or one or more other entities capable of forming chlorine dioxide in a liquid medium when exposed to acid. Thus, stabilized chlorine dioxide comprises at least one of a chlorine dioxide-containing oxy-chlorine complex, a chlorite-containing component, or an entity capable of forming chlorine dioxide in a liquid medium when exposed to acid. In the present invention, stabilized chlorine dioxide reacts with an acid, such as acetic acid and/or lactic acid, carbonic acid, or any such acid capable of reacting with the stabilized chlorine dioxide to form active chlorine dioxide. The acid may be naturally present in the fermentation system, or may be produced by contaminating bacteria, introduced into the fermentation system as part of fermentation components, or produced as a result of metabolic activity by inoculant yeast. When activated by acid, chlorine dioxide is a wide spectrum biocide, capable of eliminating the deleterious impact of the contaminating bacteria in a fermentation system. Stabilized chlorine dioxide may also be referred to as “chlorine dioxide precursor” or abbreviated herein as “SCD”.

Among the preferred chlorine dioxide-containing oxy-chlorine complex is selected from the group consisting of complex of chlorine dioxide with carbonate, complex of chlorine dioxide with bicarbonate and mixtures thereof. Examples of chlorite-containing components include metal chlorites, and in particular alkali metal and alkaline earth metal chlorites. A specific example of a chlorite-containing component which is useful as a chlorine dioxide precursor is sodium chlorite, which can be used as technical grade sodium chlorite. The exact chemical composition of many of stabilized chlorine dioxide, and in particular, chlorine dioxide complexes, is not completely understood. The manufacture or production of certain chlorine dioxide precursors is described by Gordon, U.S. Pat. No. 3,585,147 and Lovely, U.S. Pat. No. 3,591,515. Specific examples of useful stabilized chlorine dioxide include, for example, ANTHIUM DIOXCIDE, available from International Dioxcide Inc., North Kingstown, R.I.; OXINE and PUROGENE, available from Bio-Cide International, Inc., Norman, Okla.

The stabilized chlorine dioxide (chlorine dioxide precursor), SCD, may be provided in a liquid medium at a predetermined concentration, e.g., a concentration chosen to provide a disinfecting amount of chlorine dioxide in response to at least one factor other than the presence of the organic acids to be reduced. Preferably, the liquid medium has sufficient SCD so as to have a potential concentration of chlorine dioxide in the range of about 0.002% to about 40% by weight, preferably, in the range of about 2% to about 25% by weight, more preferably in the range of about 5% to about 15% by weight, based on the total weight of the liquid medium including the chlorine dioxide-containing complexes and/or one or more chlorite-containing components and/or one or more other entities capable of forming chlorine dioxide.

The stabilized chlorine dioxide may be provided as a solid material, such as a composition comprising an alkali or alkaline earth metal chlorite powder, inert ingredients, and optionally dry activator such as a dry acid. Preferably the metal chlorite is an alkali metal chlorite, more preferably sodium chlorite.

Stabilized chlorine dioxide is activated in situ by lowering pH to less than pH 8, for example by adding acid, metals and/or by in situ acid production, e.g., from certain acid-producing bacteria. Furthermore, in a fermentation system in which yeast is the inoculant, the yeast metabolize sugar and produce carbon dioxide gas (CO₂) along with ethanol and other co-products. In an aqueous environment, the CO₂ reduces the pH as a result of its conversion to carbonic acid. The lower the pH, the faster the SCD is activated. For purposes herein, the SCD remains inactive in the solution until acid is generated, which converts the SCD to activated chlorine dioxide. The more acids generated, the more activated chlorine dioxide is produced. Fermentation processes in which yeast is the inoculant are typically and deliberately carried out at pH between 3.5 to 6.5. Within this pH range, metabolic processes within the yeast cell can be sustained at an optimal level.

### Process

The present invention is a process to substantially prevent or impede the growth of undesirable microorganisms in a system wherein a nutrient source reacts with an inoculant yeast to produce a product such as ethanol. The undesirable microorganism can be contaminant yeast.

One embodiment of the process of this invention comprises introducing a nutrient source, an inoculant microorganism, and a stabilized chlorine dioxide solution into a reaction system wherein the inoculant metabolizes the nutrient source to increase in population, that is, undergo propagation. By inoculant microorganism, it is meant a microorganism deliberately selected for its ability to convert a nutrient source into a desired product of optimal quality and quantity. The inoculant microorganism can be selected from the group consisting of yeast, fungi, bacteria, and algae.

A nutrient source is a source of elements essential to the growth and survival of the microorganism. A nutrient source can comprise carbon, nitrogen, oxygen, sulfur, phosphorus, magnesium, and a variety of trace elements such as metals. Examples of nutrient sources for each of the aforenamed elements is provided hereinabove. During propagation, the inoculant microorganisms metabolize the nutrient source and subsequently increase in cell population of the microorganism. Once the nutrients are exhausted, or the inoculant microorganisms reach a target population density, subsequent separation and purification processes such as centrifugation, drying, freezing, etc, may be carried out depending on the desired end product of propagation. The inoculant microorganism can preferably be an inoculant yeast.

A process for the propagation of yeast comprises contacting a nutrient source and an inoculant yeast in an aerobic reaction system and further contacting with a stabilized chlorine dioxide solution. By “aerobic reaction system” is meant the propagation process is performed in the presence of oxygen. The preferred temperature is from about 33° C. to about 35° C. (about 92° F. and about 94° F.).

The nutrient source comprises a fermentable sugar, preferably glucose. The concentration of glucose should be no more than about 2% of the reaction system. Too high of a glucose concentration can induce the yeast to produce ethanol. The nutrient source may further comprise a nitrogen source, such as urea, which is typically present at a concentration of 300 to 500 parts per million (ppm). The nutrient source may further comprise minerals, such as magnesium and zinc.

An enzyme, such as glucoamylase is typically added to the propagation process.

Alternatively, a process of this invention comprises introducing a nutrient source, an inoculant microorganism, and a stabilized chlorine dioxide solution into a reaction system wherein the inoculant microorganism metabolizes the nutrient source and generates a by-product of the metabolic process, such as one or more enzymes. Enzymes are produced by microorganisms to facilitate metabolic reactions in cells that may not otherwise be performed in living cells. Microorganisms produce enzymes specific to the compounds from which metabolic substrates can be converted into available forms. Examples include proteases that break down proteins, and lipases that break down lipids. These processes are well known. Microorganisms can be selected and induced to produce enzymes in desired quantities.

Microorganisms such as fungi require glucose as their primary source of energy for cellular metabolism. Enzymes produced by the fungi can break down more complex compounds such as cellulose, starch, lignin, etc in order to convert these compounds into available glucose. For example, many fungi are capable of producing large quantities of enzymes such as amylases and glucosidases in fermentation processes where the nutrient source is selected from the group consisting of a fermentable sugar, nitrogen source, vitamins, trace elements and combinations thereof. By “trace elements” is meant in general, elements that are present in minute quantities in the system, but are essential for the proper growth and survival of the microorganism. Examples include manganese, zinc, iron, and copper, in addition to, or including sulfur, phosphorus, magnesium. Thus, fungi can be used as inoculant microorganisms, and the enzyme produced can be separated and purified for use in other processes.

In a particular embodiment of this invention, there is a process to substantially impede the growth of contaminant yeast in a fermentation system, without similarly impeding the growth of inoculant yeast, comprising introducing a fermentable sugar, an inoculant yeast, and a stabilized chlorine dioxide solution into a fermentation system wherein the inoculant converts the sugar into ethanol and carbon dioxide. The stabilized chlorine dioxide is added to one or more of the fermentable sugar, the inoculant, or other operations of the fermentation system.

The following is a description of how a process of this invention may be performed, when corn is the feedstock in fuel ethanol production. It will be understood by those skilled in the art this process may be varied, e.g., by use of other feedstocks, such as molasses, and applied to beverage ethanol production or other biological production processes where contamination may arise.

The process to produce ethanol comprises fermenting a sugar by contacting the sugar with an inoculant yeast, in a fermentation system, to produce a fermentation product comprising ethanol and carbon dioxide. The process can be batch or continuous. By “fermentation system” it is meant herein to refer to a system comprising a batch- or continuous-flow liquefaction vessel, fermentation vessel, heat exchanger, and piping, in which the introduction of fermentation reactants (yeast, fermentable sugar) are introduced, and subsequent fermentation of sugar occurs. By liquefaction, it is meant the process by which sugars are physically and chemically modified to a form which can be readily metabolized by inoculant yeast. The fermentation vessel can be a tank or other reactor form, including a plug flow reactor into which components of the fermentation (inoculant, fermentable sugar, nutrients, SCD, etc) are introduced and fermentation takes place over time. In the process of this invention, stabilized chlorine dioxide may be added to the fermentable sugar or the inoculant yeast prior to their introduction into the fermentation system. Alternatively or in addition, SCD may be added as a separate stream to the fermentation system, apart from the fermentable sugar and inoculant. SCD is added in an amount effective to substantially prevent growth of undesirable microorganisms in the system.

In a batch process, the SCD may be added before, during and/or following the addition of the fermentable sugar and/or inoculant yeast to the fermentation system. SCD may be added to a yeast propagation tank. Preferably, the SCD will be added before addition of fermentable sugar or before addition of inoculant yeast to the fermentation system to gain the best results. Most preferably, the SCD is added before addition of the inoculant yeast.

Stabilized chlorine dioxide (SCD) is added to one or more of the fermentable sugar, the inoculant yeast, or the fermentation system to control establishment and growth of contaminant yeast in the system. Stabilized chlorine dioxide can be added to prevent growth and establishment of contaminant yeast in the system. That is, stabilized chlorine dioxide is added prior to substantial accumulation or growth of contaminant yeast in the system, such as prior to the introduction of any or all of the ingredients necessary to initiate the fermentation process. Alternatively, stabilized chlorine dioxide can be added to the fermentation system in an amount effective to substantially reduce the population of contaminant yeast that has become established in the system, while having little or no deleterious effect on growth and establishment of inoculant yeast.

SCD is added to control contaminant yeast from growing in the fermentation system. Surprisingly, SCD remains in the fermentation system and although contaminant yeast are adversely affected, there is little effect on the desirable (inoculant) yeast through in situ generation of activated chlorine dioxide, that is, ClO₂, by reaction of SCD with acid produced in the system.

SCD is added in an amount effective to substantially prevent growth of contaminant yeast, but have negligible impact, including negligible deleterious impact, on the fermentation process. By “effective amount” is meant an amount that is capable of substantially inhibiting the growth, or reducing the viable population of contaminant yeast without adversely affecting the fermentation process. By “substantially inhibit the growth of contaminant yeast,” it is meant that viable population of contaminant yeast does not increase by more than 10-fold following exposure to “the application.” By “substantially reducing the viable population of contaminant yeast”, it is meant that the viable population of contaminant yeast decreases by at least 10-fold, following exposure to “the application.” Such conditions allow the inoculant yeast to quickly and effectively convert fermentable sugar to ethanol. By operating a fermentation plant in accordance with this invention, a reduced rate in frequency of, with potential elimination of, contaminant yeast contamination is achieved. Thus, in the process of this invention, long term productivity and profitability increase in the operation of a fermentation plant.

SCD is added to the fermentation system or to a feed to the fermentation system to the fermentation system or both at concentrations of about 0.0001% (1 parts per million, ppm) to about 5% (50,000 ppm), based on the weight of activated chlorine dioxide that can be produced and the total weight of the contents of the fermentation system. Preferably the amount of stabilized chlorine dioxide added is about 0.003% (30 ppm) to about 0.1% (1000 ppm), more preferably 0.005% (50 ppm) to 0.04% (400 ppm) of the total volume of material in the fermentation vessel. The amount of stabilized chlorine dioxide is sufficient to minimize process interruptions due to undesired yeast contamination and to eliminate the need for other biocide or antibiotic.

Stabilized chlorine dioxide is added to the system at pH levels not greater than 6.5, these conditions being sufficient to prevent growth of contaminant yeast without affecting the viability of the inoculant yeast.

The product mixture from the fermentation system comprises ethanol, water, inoculant yeast and un-reacted SCD. After discharge from the fermentation system, conventional process steps for separation and purification or other processing of the ethanol may be performed. Subsequent steps can include (a) distilling the fermentation product to remove about 95% of the liquid, as well as the solids and produce a distilled ethanol comprising about 5% water; and (b) dehydrating the distilled ethanol, for example using molecular sieves, thereby producing 100% (200 proof) ethanol. Additional steps for producing industrial ethanol can comprise (c) denaturing the dried ethanol by mixing in about 2-5% gasoline or other additive; and (d) recovering co-produced carbon dioxide and solids. Additional steps in beverage ethanol production may include (c) aging, (d) blending, and (e) bottling the product such as those described in U.S. Patent Application 20060159812A1. Beverage ethanol production is also described in Kirk-Othmer Encyclopedia of Chemical Technology, “Beverage Spirits, Distilled” by John E. Bujake, John Wiley & Sons, Inc. (New York), 2001. Additional steps can include recovering one or both of co-produced carbon dioxide and solids. These processes are known to those skilled in the art.

When the feedstock for the fermentable sugar is corn, comprises ethanol, water, inoculant yeast, and grain solids. This product mixture may be distilled to separate the ethanol from the bulk of the water present and from the solids (which include inoculant yeast and grain solids). The solids may be recovered. The recovered solids can be used in animal feed and mixed with distiller grains.

When the feedstock for the fermentable sugar is molasses the product mixture may be distilled to separate the ethanol from the bulk of the water present and from the solids. The solids obtained from molasses comprise mainly spent yeast cells (yeast cream), which can be re-used in subsequent fermentations after being conditioned in a process commonly referred to as washing. Other compounds may be introduced in order to prepare the yeast cream for subsequent fermentation. These steps are also known to those skilled in the art.

By operating a fermentation plant in accordance with this invention, a reduced rate in frequency of, with potential elimination of, deleterious effects of contaminating microorganisms, such as bacterial infection and growth of undesirable yeast, are achieved. Thus, in the process of this invention, long term productivity and profitability increase in the operation of a fermentation plant.

It is recognized that individual results at individual ethanol fermentation plants operating under different conditions may vary in the relative improvements in the process of this invention in the control of and reduction of undesirable microorganisms such as bacteria and contaminant yeast, and increases in ethanol production relative to absence of treatment with stabilized chlorine dioxide, relative to the absence of SCD or relative to the addition of remedial SCD.

### EXAMPLES

In the following examples, the stabilized chlorine dioxide that was used was ANTHIUM DIOXCIDE, available from International Dioxcide Inc., North Kingstown, R.I., as a solution containing 5% chlorine dioxide when activated.

Total viable cells in the samples herein was measured as a concentration of colony forming units (CFU) per unit of volume (i.e., CFU/ml) or per unit of mass (i.e., CFU/g) of sample, or based on optical density readings using a spectrometer. Optical density as measured using the spectrophotometer represents the amount of light of specific wavelength (600 nm) absorbed by yeast cells and is directly proportional to the concentration of yeast in the sample. That is to say, the higher the concentration of cells in the suspension, the higher the optical density of the sample, and vice versa. When used to compare yeast cells exposed to varying conditions, lower optical densities indicate inhibition of yeast growth. It is also understood that there is a direct correlation of concentration of viable cells in the samples and the CFU measurement. Thus, the higher the concentration of yeast, the higher the CFU and vice versa. As a convention, CFUs are transformed mathematically into logarithmic values (Log₁₀CFU) to simplify comparisons between different treatments.

### Example 1

In this example, stabilized chlorine dioxide was used to inhibit a contaminant strain of the yeast Saccharomyces cerevisiae. Undesired S. cerevisiae are common contaminants in industrial ethanol.

Fermentation samples (1 liter) were collected from primary fermentation tanks of an ethanol plant operating in continuous mode using fractionated corn as a feedstock. The plant had been experiencing a drop in their final ethanol production levels and an increase in their residual sugars. The fermentation samples were collected from the plant and shipped to a testing site on ice. At the testing site, the samples were diluted using sterile phosphate-buffered saline (available from Sigma, St. Louis, Mo.) and plated (0.1 ml) onto the surface of WL Nutrient agar plates (available from Becton, Dickinson and Co., Franklin Lakes, N.J.). Plates were incubated at 32° C. overnight and individual colonies of yeast were then streaked for isolation.

Samples of the dry yeast used at the plant (Safdistil C-70, Lesaffre, Marcq en Baroeul, France) were hydrated by placing into sterile warm water and also plated onto WL Nutrient agar plates. Again, plates were incubated at 32° C., and individual colonies were then streaked for isolation.

The fermentation samples gave rise to plates that predominantly contained dark green colonies on WL Nutrient agar. Samples of the dry yeast resulted in cream-colored colonies with dark rings around them. This indicated that the fermentation system was grossly contaminated with an undesired yeast strain. The two different yeast strains were typed using DNA fingerprinting techniques and results demonstrated that the contaminant yeast was also a strain of S. cerevisiae. Thus, the primary fermentation vessels at the plant were grossly contaminated with an undesired strain of Saccharomyces cerevisiae, as determined by visual inspection of the colonies on the plates and by using DNA fingerprinting.

Single colonies of both the dry yeast and the contaminant S. cerevisiae were each used to inoculate tubes containing 15 milliliters of Yeast Peptone Dextrose broth (available from Becton, Dickinson and Co.) and incubated overnight at 32° C. Twenty five milliliters of fermentation sample were dispensed into 50 ml polypropylene tubes (VWR, Bridgeport, N.J.) and 2.5 ml of overnight cultures of yeasts were added to the tubes. Various amounts of stabilized chlorine dioxide were added to each tube to give final concentrations of 0, 90, 180, and 270 parts per million. Dilutions of each sample were prepared in sterile phosphate-buffered saline and 0.1 ml plated onto WL Nutrient agar plates. The pH of the fermentation sample is around 4.0-4.2. Plates were incubated overnight at 32° C. Tubes were then incubated for 2 hours at 32° C. and samples were again plated onto WL plates. Results are provided in Table 1.

TABLE 1
Response of S. cerevisiae Safdistil C-70 and contaminant S. cerevisiae
to stabilized chlorine dioxide in fractionated corn mash

Sample Time

| Yeast | 0 hour | 2 hour |
|---|---|---|
| Contaminant S. cerevisiae control | 5.85 | 5.95 |
| Contaminant S. cerevisiae 90 ppm | 5.95 | 5.85 |
| Contaminant S. cerevisiae 180 ppm | 6.18 | 5.4 |
| Contaminant S. cerevisiae 270 ppm | 6.72 | 2.40 |
| S. cerevisiae C-70 control | 6.57 | 5.90 |
| S. cerevisiae C-70 90 ppm | 6.93 | 6.84 |
| S. cerevisiae C-70 180 ppm | 6.91 | 6.43 |
| S. cerevisiae C-70 270 ppm | 6.74 | 5.00 |

Results reported as log CFU/ml.

As can be seen in Table 1, both yeasts were inoculated into the fermentation samples at approximately 1,000,000 colony forming units per milliliter (6 log CFU/ml). Control samples received no treatment with stabilized chlorine dioxide, while test samples received 90, 180, or 270 ppm. Samples were plated immediately upon addition of stabilized chlorine dioxide, and again after incubation at 32° C. for 2 hours. Samples receiving 90 ppm of stabilized chlorine dioxide show little difference in the initial load of contaminant S. cerevisiae or the Safdistil C-70 yeast and in the concentration of these two yeasts after two hours of incubation. This is expected since yeasts generally have a high tolerance to treatment with stabilized chlorine dioxide. At 180 ppm, the population of contaminant S. cerevisiae is reduced by approximately 0.8 log unit (6.18 to 5.4 log CFU/ml), while the Safdistil C-70 yeast appears more resistant to treatment, with a reduction of less than half of one log unit. The data from treatment with 270 ppm of stabilized chlorine dioxide show, the contaminant S. cerevisiae is reduced from 6.72 log CFU/ml at time zero to 2.39 log CFU/ml (a reduction of 4.3 log) at the two hour time point, while the Safdistil C-70 yeast is only reduced by approximately 1.7 log units. This difference in susceptibility to stabilized chlorine dioxide can be used to selectively inactivate contaminant yeast, with little effect on the desired fermenting strain (inoculant yeast).

### Example 2

In this example, the responses of S. cerevisiae and Pichia fermentans to stabilized chlorine dioxide were determined. S. cerevisiae is the most common inoculant yeast used in ethanol production. In a recent survey, members of the genus Pichia were often found in primary fermentation vessels of contaminated ethanol production facilities. This Example determined whether desirable S. cerevisiae and undesirable P. fermentans yeasts responded differently when challenged with various levels of stabilized chlorine dioxide.

Safdistil C-70 S. cerevisiae was again utilized for this example as the desirable inoculant yeast. The contaminant yeast, P. fermentans ATCC 10136 is available from the American Type Culture Collection, Manassas, Va. Isolated colonies from plates containing S. cerevisiae Safdistil C-70 and P. fermentans ATCC 10136 were used to inoculate 15 milliliter tubes containing 10 milliliters of Yeast Peptone Dextrose broth (Becton, Dickinson and Co.). Cultures were incubated overnight at 32° C. with agitation. Cultures were diluted 1 to 100 and inoculated into 96-well microtiter plates (Becton, Dickinson and Co.) containing stabilized chlorine dioxide at concentrations ranging from 0-375 parts per million. Optical density (OD) readings at 600 nanometers (600 nm) were recorded at two hour intervals for 23 hours using an automated microtiter plate reader set to incubate at 32° C. (PowerWave microplate spectrophotometer, available from Bio-Tek Instruments, Inc., Winooski, Vt.). Optical density is an indication of the concentration of yeast cells within an individual well. The higher the turbidity within an individual well indicates a higher concentration of yeast cells.

TABLE 2
Response of S. cerevisiae C-70 and Pichia fermentans ATCC 10651
to treatment with stabilized chlorine dioxide

| | Stabilized Chlorine Dioxide dose | | | | |
|---|---|---|---|---|---|
| | 0 ppm | 7.5 | | 37.5 | |
| Yeast/Time of result | (Control) | ppm | 15 ppm | ppm | 75 ppm |
| Pichia fermentans ATCC | 0.104 | 0.104 | 0.105 | 0.107 | 0.102 |
| 10651-0 hour | | | | | |
| Pichia fermentans ATCC | 0.196 | 0.19 | 0.199 | 0.197 | 0.152 |
| 10651-8 hour | | | | | |
| Pichia fermentans ATCC | 0.445 | 0.416 | 0.423 | 0.345 | 0.156 |
| 10651-16 hour | | | | | |
| Pichia fermentans ATCC | 0.792 | 0.726 | 0.718 | 0.484 | 0.158 |
| 10651-23 hour | | | | | |
| S. cerevisiae C-70-0 hour | 0.111 | 0.104 | 0.102 | 0.102 | 0.102 |
| S. cerevisiae C-70-8 hour | 1.558 | 1.547 | 1.551 | 1.504 | 0.606 |
| S. cerevisiae C-70-16 hour | 1.849 | 1.85 | 1.849 | 1.848 | 1.582 |
| S. cerevisiae C-70-23 hour | 1.854 | 1.854 | 1.854 | 1.854 | 1.869 |

Results are reported as absorbance at 600 nm.

As can be seen in Table 2, S. cerevisiae and P. fermentans respond very differently to the challenge of 37.5 ppm of stabilized chlorine dioxide. In the presence of 37.5 ppm stabilized chlorine dioxide, S. cerevisiae grows extremely well, reaching an optical density of 1.504 units after just 8 hours. At 16 and 23 hours, the optical density of the control sample of S. cerevisiae is the same as those samples treated with 7.5, 15, and 37.5 ppm. Even at 75 ppm stabilized chlorine dioxide, there is no difference in the optical density at 23 hours compared to the control. P. fermentans is not as resistant to treatment with stabilized chlorine dioxide compared to S. cerevisiae. P. fermentans exhibited a decrease of approximately 0.3 OD (optical density) units between the sample treated with 37.5 ppm stabilized chlorine dioxide and the control at 23 hours.

These results demonstrate that stabilized chlorine dioxide at 37.5 ppm is able to differentially inhibit Pichia fermentans ATCC 10631, while having little or no effect on S. cerevisiae.

### Example 3

The response of Saccharomyces cerevisiae to treatment with stabilized chlorine dioxide was compared with the response of two species of Dekkera and two species of Pichia, both of which are common wild yeast contaminants (contaminant yeast) of ethanol production. Safdistil C-70 S. cerevisiae was again used for this example. Dekkera bruxellensis FDFD 268, Dekkera anomala FDFD 262, Pichia fermentans ATCC 10651, and Pichia (Hansenula) jadinii FDFD 168 were selected. Yeasts were prepared by inoculating tubes containing 5 milliliters Yeast Peptone Dextrose broth and incubating overnight at 32° C. with agitation. Molasses medium was prepared by adding 178 grams of molasses, 5 grams of brown sugar, 1 gram of yeast extract, 2 grams of potassium phosphate, and 5 grams of urea to 810 milliliters of water. The molasses medium was adjusted to pH 5.0 using sulfuric acid. Twenty milliliters of molasses medium was dispensed into 50 milliliter conical tubes, followed by 200 microliters of yeasts grown overnight in Yeast Peptone Dextrose broth. The tubes received a dose of 112.5 parts per million stabilized chlorine dioxide and were then incubated at 32° C. Samples were dilution-plated onto Potato Dextrose Agar (Becton, Dickinson and Co.) at 2 and 24 hours post inoculation. Results can be seen in Table 3.

TABLE 3
Response of S. cerevisiae C-70 and contaminant yeasts to treatment
with stabilized chlorine dioxide in molasses medium

| | Treatment | | | |
|---|---|---|---|---|
| | 2 hour | 2 hour | 24 hour | 24 hour |
| | control | treated | control | treated |
| | (no SCD | (112.5 ppm | (no SCD | (112.5 ppm |
| Yeast | treatment) | SCD) | treatment) | SCD) |
| S. cerevisiae C-70 | 5.99 | 5.98 | 9 | 8.73 |
| Dekkera | 5.59 | 5.62 | 8.39 | <1 |
| bruxellensis | | | | |
| FDFD 268 | | | | |
| Dekkera anomala | 5.28 | 3.99 | 6.83 | <1 |
| FDFD 262 | | | | |
| Pichia fermentans | 5.35 | 5.46 | 8.31 | 5.45 |
| ATCC 10651 | | | | |
| Pichia jadinii | 5.94 | 5.86 | 8.78 | 5.64 |
| FDFD 168 | | | | |

Results are reported as log CFU/ml after treatment.
Samples treated with 112.5 ppm stabilized chlorine dioxide (SCD).

As can be seen in Table 3, two hours of treatment with 112.5 ppm resulted in negligible reductions of Safdistil C-70 S. cerevisiae C-70, D. bruxellensis FDFD 268, P. fermentans ATCC 10651, and P. jadinii FDFD 168 compared to untreated control samples. The numbers of D. anomala FDFD 262 were reduced by approximately 1.29 log units in two hours after treatment with 112.5 ppm stabilized chlorine dioxide. After 24 hours, all of the yeasts in the control (untreated) samples were able to grow from an initial inoculum of approximately 5 log CFU/ml to approximately 8-9 log CFU/ml, except for D. anomala which only reached a level of 6.83 log CFU/ml after 24 hours of incubation. The 24-hour treated sample of S. cerevisiae C-70 was resistant to treatment with 112.5 ppm stabilized chlorine dioxide as seen by the <0.3 log difference in the populations of S. cerevisiae in the control and treated samples. The 24-hour treated samples of D. bruxellensis FDFD 268 and D. anomala FDFD 262 were reduced to below the detectable limit when treated with 112.5 ppm stabilized chlorine dioxide. The 24-hour treated samples of P. fermentans ATCC 10651 and P. jadinii FDFD 168 were unable to grow in the presence of 112.5 ppm stabilized chlorine dioxide, since the level of yeasts in the treated samples were similar to those in the 2-hour control samples.

These results indicate that yeasts of the genus Dekkera and Pichia are more sensitive to stabilized chlorine dioxide than S. cerevisiae.

What is claimed is:

1. A process to substantially impede growth of contaminant yeast in a fermentation system comprising introducing a fermentable sugar, an inoculant yeast, and a stabilized chlorine dioxide solution into a fermentation system wherein the inoculant yeast converts the sugar into ethanol and carbon dioxide.

2. The process of claim 1 wherein the stabilized chlorine dioxide is added to the fermentable sugar.

3. The process of claim 1 wherein the stabilized chlorine dioxide is added to the inoculant yeast.

4. The process of claim 1 wherein the fermentation system comprises a batch- or continuous-flow liquefaction vessel, fermentation vessel, heat exchanger, and piping, and wherein the stabilized chlorine dioxide is added to the fermentation vessel.

5. The process of claim 1 wherein the stabilized chlorine dioxide is added in an amount from about 0.0001% (1 ppm) to about 5% (50,000 ppm), based on the weight of activated chlorine dioxide which can be produced and total weight of the contents of the fermentation vessel.

6. The process of claim 5 wherein the stabilized chlorine dioxide is added in an amount from about 0.003% (30 ppm) to about 0.1% (1000 ppm), based on the weight of activated chlorine dioxide which can be produced and total weight of the contents of the fermentation vessel.

7. The process of claim 1 wherein the stabilized chlorine dioxide is a chlorine dioxide-containing oxy-chlorine complex which is a complex of chlorine dioxide with carbonate, chlorine dioxide with bicarbonate or a mixture thereof.

8. The process of claim 1 wherein the stabilized chlorine dioxide is a chlorite-containing component, which is one or more metal chlorites.

9. The process of claim 8 wherein the metal chlorite is sodium chlorite.

10. The process of claim 1 wherein the stabilized chlorine dioxide is provided in a liquid medium at a concentration having a potential concentration of activated chlorine dioxide in the range of about 0.002% to about 40% by weight, based on the total weight of the liquid medium.

11. The process of claim 10 wherein the stabilized chlorine dioxide is provided in a liquid medium at a concentration having a potential concentration of activated chlorine dioxide in the range of about 2% to about 25% by weight, based on the total weight of the liquid medium.

12. The process of claim 1 wherein the inoculant is added in an amount of about 1 pound of dry yeast per 1000 gallons (1 kilogram per 8000 liters) of composition comprising fermentable sugar.

13. The process of claim 1 wherein the fermentable sugar is derived from one or more of one or more corn, wood chips, wheat straw, corn stover, switch grass, milo, barley, millet, sorghum, sugar cane, sugar beets, molasses, whey, potatoes.

14. The process of claim 13 wherein the fermentable sugar is derived from corn.

15. The process of claim 14 wherein the fermentable sugar is present in the fermentation system in a concentration of about 5 to about 40% (weight/volume).

16. The process of claim 1 wherein the fermentable sugar is derived from corn and is present in the fermentation system in a concentration in the range of about 10 to 35% (weight/volume); the stabilized chlorine dioxide is added to the fermentation system before addition of fermentable sugar or before addition of inoculant to the fermentation system; the stabilized chlorine dioxide is added in an amount from about 0.005% (50 ppm) to about 0.04% (400 ppm), based on the weight of activated chlorine dioxide which can be produced and total weight of the contents of the fermenter; the stabilized chlorine dioxide is an alkali metal chlorite or an alkaline earth metal chlorite, which is provided in a liquid medium at a concentration having a potential concentration of activated chlorine dioxide in the range of about 2% to about 25% by weight, based on the total weight of the liquid medium.

17. A process to substantially impede growth of contaminant microorganism in a reaction system for production of a product derived from the metabolism of a nutrient source by an inoculant microorganism comprises introducing an inoculant microorganism and a stabilized chlorine dioxide solution into the reaction system.

18. The process of claim 1 wherein the nutrient source is selected from the group consisting of a fermentable sugar, nitrogen source, vitamins, trace elements and combinations thereof, the inoculant microorganism is a fungi and wherein the product is an amylase or glucosidase.

19. A process for the propagation of yeast comprising contacting a nutrient source and an inoculant yeast in an aerobic reaction system and further contacting with a stabilized chlorine dioxide solution.

20. The process of claim 19 wherein the temperature is from about 33° C. to about 35° C. (about 92° F. and about 94° F.), the nutrient source comprises glucose, at a concentration of no more than about 2% of the reaction system, and urea, at a concentration of a rate of 300 to 500 parts per million (ppm).
